(19) United States
(12) Patent Application Publication
(10) Pub. No.: US 2006/0045852 A1
(43) Pub. Date: Mar. 2, 2006

(54) Materials and methods for tooth surface preparation for dental bonding

(76) Inventor: Gerard T. Ortner JR., Rancho Murieta, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 10/928,604

(22) Filed: Aug. 26, 2004

(51) Int. Cl. A61K 8/66; A61K 8/40

(52) U.S. Cl. 424/49; 424/50

(57) ABSTRACT

Surface conditioner compositions and methods for hemostatic control are provided for preparing tooth surfaces for bonding with dental restorations such as crowns or veneers. One embodiment of the composition includes an etchant and at least one hemostatic agent. A second embodiment of the composition includes an etchant, a hemostatic agent and an antifibrinolytic agent. Hemostatic control may also be provided with the use of a hemostatic agent or an antifibrinolytic agent in a try in or evaluation paste used to position the restoration for evaluation prior to applying a permanent adhesive. The compositions may also include a colorant.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to dental preparations for humans or animals, and more particularly to a topical dental preparation that includes an etchant and at least one bleeding inhibitor for use in conditioning and preparing teeth for bonding with a crown or other dental restoration such as a veneer. Although the preparation and methods of the invention are particularly suited to for preparation of tooth surfaces for bonding, it will be understood that the invention may be suitable for any bone or other biological substrate that may require the application of an adhesive and will benefit from surface preparation with an etchant.

2. Description of Related Art

Historically, various metals and ceramic materials have been used to produce prosthetic replacements for teeth. Many early tooth replacements were retained in the mouth by being mechanically locked into position with the use of shaped holes or grooves. Such replacements required the removal of healthy material from the body of the tooth and therefore generally weakened the structure of the base tooth.

Advancements in dental procedures have seen a shift from primarily the use of metal restorations to the use of non-metal materials bonded with adhesives permitting the conservation of existing tooth structures. Biologically inert adhesives and cements have allowed, essentially, the permanent placement of medical and dental appliances in the human body without requiring significant tooth or bone removal. However, the success or permanence of the bond provided by an adhesive will depend in part on the condition of the surface of the tooth that is to receive the crown or other dental appliance. Proper preparation of the tooth surfaces prior to the application of an adhesive is essential for strong bonding between tooth surfaces and a dental prosthetic.

One common use of a dental adhesive is with the installation of a crown or veneer. Crowns are often used to repair broken, misaligned, discolored or badly decayed teeth and allow a restoration to the natural size, shape and function of the original teeth. Crowns may also provide relief to sensitive teeth that are painful when exposed to hot and cold temperatures or pressure due to microscopic cracks that are present in the teeth without removing the teeth. Teeth that have a high risk of fracture or have sustained a significant loss of structure due to fracture or caries are appropriate candidates for dental crowns.

Normally, the tooth is prepared for the crown by reducing its size and shape so that it is suitable to have a crown placed over the top of the tooth and permanently cemented into place. The typical crown installation takes place during two visits with the dental practitioner. During the first visit, the dentist will re-contour the tooth receiving the crown by removing a thin layer of tooth structure from the chewing surface and the sides of the tooth. The amount of tooth structure that is removed will depend on the type of crown that is used and is usually within the range of 1 mm to 2 mm of tooth material. An impression of the reduced tooth is then taken to permit the fabrication of a permanent crown specifically tailored to the dimensions of the reduced tooth. A temporary crown is generally placed over the reduced tooth with temporary cement to protect the reduced tooth until the permanent crown can be fabricated.

After the permanent crown is fabricated, the patient returns for a second visit to have the temporary crown removed by the dental practitioner and a permanent crown installed. The surface of the tooth is prepared for placement of the cement and the installation of the permanent crown. The strength of the cement bond and the longevity of the permanent crown are dependent on the proper preparation of the surface of the tooth and crown. The junction between the cement layer disposed between the tooth and the crown must be free of contaminants in order to maximize the bonding strength of the cement.

It has been observed that a layer forms under the temporary crown that may be composed of blood and saliva components, dentin and enamel debris, oils, castings from rotary dental instruments and the like. In addition, the removal of the temporary crown, final tooth preparation and preliminary placement of the permanent crown may cause bleeding from the gums. These contaminants can interfere with the cement bond between the tooth and the restoration. Further preparation of the enamel surface of the tooth for bonding may include abrasion of the tooth surfaces with a dental bur or pulsed lasers. Elimination of the contaminants after removal of the temporary crown is usually attempted with pressurized water and air. If the permanent crown or veneer is placed without removing the contaminants, the cement seal may be compromised over time due to leakage on the microscopic level in the junction.

In addition, the interior of the tooth is composed of dentin. Human dentin is generally composed of a solid circumpulpal phase surrounding a network of tubules. These tubules, measuring about 1 to 3 micrometers in diameter, contain elongated cell bodies that radiate from the pulp throughout the entire dentin. Structurally dentin is composed of mineral crystals deposited between a network of protein fibrils. Dentin contains approximately 70% polycrystalline calcium hydroxyapatite by weight. Achieving a strong durable bond with human dentin with bonding agents is often difficult because of the structure of dentin. The lack of adhesion between dentin and prosthetic material may also result in the formation of marginal gaps creating interfacial leakage, which can lead to tooth discoloration and secondary cavities.

It can be seen that the ability to achieve a strong durable bond to human dentin and enamel through the use of bonding agents is an essential part of dental restorative techniques. There is a need for pre-bonding surface preparations and procedures that will prepare the dental surfaces or other biological substrates for permanent chemical bonding between the tooth and the dental appliance that will control contamination from gingival bleeding. The present invention satisfies this need as well as others and generally overcomes the limitations of the prior art.

### BRIEF SUMMARY OF THE INVENTION

The present invention is a dental etchant composition and method of use for providing contamination free, de-mineralized surfaces for the application of adhesives to bond prosthetics to teeth or other substrates. Surface preparations or conditioners that are applied to enamel and dentin surfaces will enhance the bonding characteristics of the adhesive with the tooth. One drawback of prior art compositions and methods is that saliva and blood from the gingiva can contaminate the surfaces of the contoured tooth. Contamination of the surface that receives the adhesive can compromise the seal between the prosthesis and the tooth as well as cause discoloration through translucent prosthetic restorations. The composition and methods of the present invention generally provide control over blood and other contaminants that may interfere with proper bonding as well as optimizing bonding by preparing the tooth surface with an etchant.

By way of example, and not of limitation, the invention includes a composition and method for preparing a tooth surface for bonding with a dental or orthodontic prosthesis comprising an etchant and at least one hemostatic agent. According to another aspect of the invention, a topical composition is provided that comprises an etchant; a first hemostatic agent and at least one second hemostatic agent.

According to another aspect of the invention, a topical surface conditioner composition for improving adhesive bonding with a dental or orthodontic appliance is provided comprising an etchant, a hemostatic agent and an antifibrinolytic agent. One embodiment provides an etchant composition that includes a colorant that identifies the presence of the liquid or gel composition when it is applied to the surface of the tooth to give a visual indication of the coverage and amount of composition that has been applied. The compositions may also include a buffer to maintain etchant conditions and minimize pH fluctuations during use.

Another embodiment of the invention provides an evaluation or “try-in paste” that includes a hemostatic agent. Try-in pastes allow the dental practitioner to orient and position the prosthesis and evaluate and match the color and alignment of the prosthesis with the adjoining teeth as well as the bite characteristics with the restoration temporarily in place. The hemostatic agent or agents that are in the try-in paste can be used in conjunction with the same or other hemostatic agents in the etchant to provide an overall scheme for controlling and eliminating the presence of blood and other contaminants on the bonding surfaces of the tooth. In one embodiment of the invention, the try-in pastes include a colorant in a variety of different shades. Different shades of colorant in the try-in pastes allow the dental practitioner to approximate the final color of translucent restorations and select a colored adhesive color that will produce predictably the desired shade.

The etchant composition according to the invention can be dispensed in liquid or gel formulations. In one embodiment, a dual chambered syringe is provided that contains the hemostatic agent in one chamber and the etchant in the other and is configured to mix the materials at the time of dispensing. In one embodiment the hemostatic agent contained one colorant and the etchant was a second, preferably complimentary color so that the combination produces a third color. Another embodiment includes a triple chambered syringe with different hemostatic agents and an etchant in each chamber. The chambers may also vary in size to accommodate variable volumes and concentrations of hemostatic agents and etchants. In another embodiment, all of the materials are pre-mixed and dispensed in a single chambered syringe. Yet another embodiment provides a syringe that will allow the materials to be dispensed separately.

The preferred hemostatic agents include epinephrine, thrombin and amino caproic acid alone or in combination. Epinephrine is a vasoconstrictor and limits the absorption of epinephrine into the system as well as limiting blood flow. Thrombin clots fibrinogen within the blood directly thereby limiting blood flow. Amino caproic acid is an inhibitor of fibrinolysis and acts to control bleeding by inhibiting plasmin, which in turn metabolizes fibronectin.

An object of the invention is to provide a method for controlling bleeding during surface preparation of a tooth surface for bonding.

Another object of the invention is to provide a composition that has at least one hemostatic agent and an etchant to allow for a protein free bonding surface.

A further object of the invention is to provide a composition that can include a vasoconstricting agent and an antifibrinolytic agent.

Another object of the invention is to provide a hemostatic composition and method for controlling pulpal and gingival bleeding to secondary symptoms from pulpal space encroachments.

Yet another object of the invention is to provide a composition that is inexpensive to manufacture, easy to use and effective.

Further objects and aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWINGS

The invention will be more fully understood by reference to the following drawings which are for illustrative purposes only:

FIG. 1 is a flow diagram of one embodiment of the method of use of the surface preparation according to the present invention adapted for use with the installation of a crown.

FIG. 2 is a flow diagram of one embodiment of the method of use of the surface preparation according to the present invention adapted for use with the installation of a veneer.

### DETAILED DESCRIPTION OF THE INVENTION

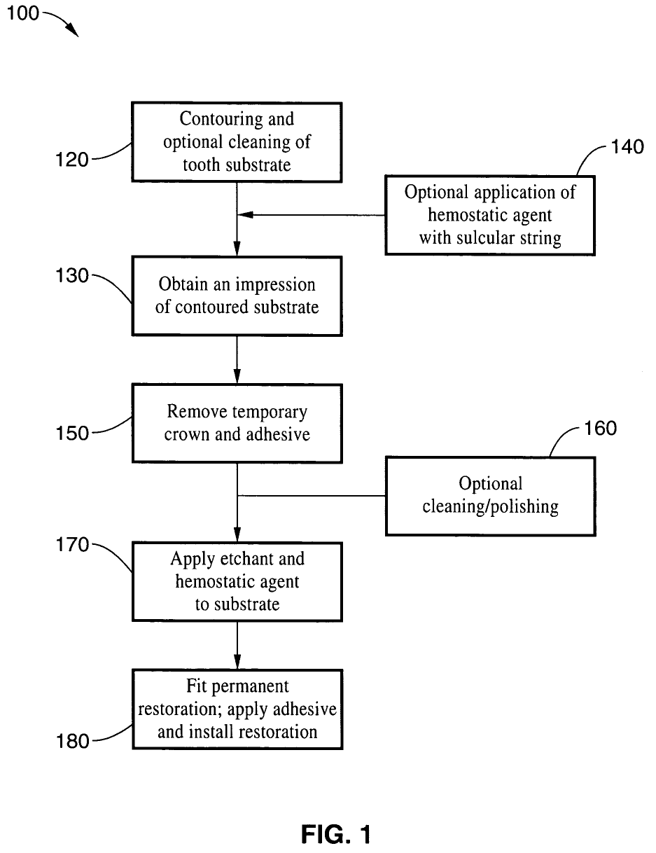

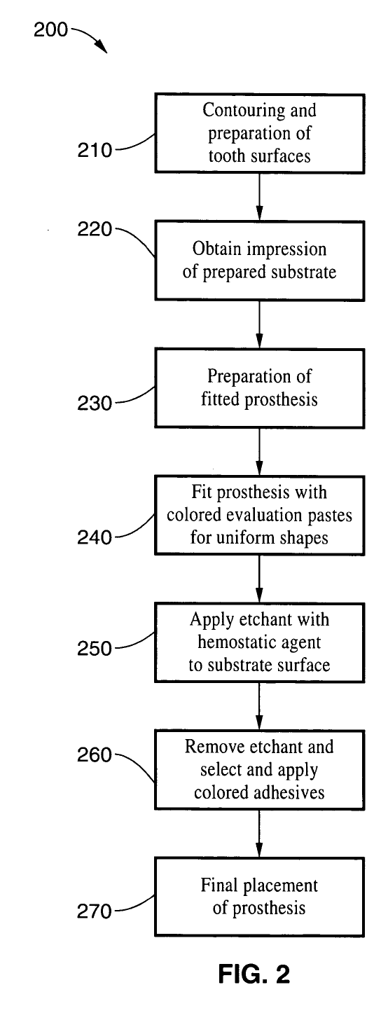

Referring more specifically to the drawings, for illustrative purposes the present invention is embodied in the compositions and methods generally shown in FIG. 1 and FIG. 2. It will be appreciated that the composition may vary as to configuration and as to details of the parts, and that the method may vary as to the specific steps and sequence, without departing from the basic concepts as disclosed herein.

The success or permanence of a dental restoration depends primarily on the strength of the bond between the tooth and adhesive. The invention provides surface conditioning compositions and methods of use that can result in improved tooth-to-adhesive bond strengths while avoiding discoloration, microleakage, degradation and secondary caries that require the repair or replacement of the restoration.

The compositions of the invention 100 for tooth surface conditioning with hemostatic control and methods of use are generally shown and illustrated by the installation of a crown in FIG. 1 and a veneer in FIG. 2. Crown and veneer installations require the shaping, contouring and reduction in size of the tooth surface to permit the permanent placement of a fixed prosthetic restoration. The contoured surface is prepared to receive the adhesive and the final installation of the restoration with the use of a composition that includes an etchant and at least one hemostatic agent. Other hemostatic controls may also be used in conjunction with the etchant composition.

Turning now to FIG. 1, contouring and other preparation of the tooth base is conducted at block 120 of FIG. 1. In the case of a crown, an impression of the shaped tooth is preferably taken and incorporated into the permanent crown to provide a crown with a close fitting socket at block 130 of FIG. 1. A socket matched to the contoured tooth can provide a close fitting union between the prosthesis and the tooth base. The preparation of a tooth for a crown or other prosthetic restoration is often at a level on the tooth that is below the gum line. Preparation of tooth surfaces below the gum line may require the retraction of gum tissue so that a proper impression can be taken. However, retraction of gum tissue and preliminary cleaning may also result in bleeding of the gums.

In one embodiment, a cord impregnated with epinephrine or other hemostatic agent is packed into the sulcus around the tooth to control any bleeding and shrink the tissue back away from the tooth at block 140 of FIG. 1. This allows the impression material to accurately duplicate the prepared margin of the preparation at block 130 of FIG. 1 for the lab technician to fabricate a precisely fitted permanent restoration. In this embodiment, a temporary crown may be made from the impression of the prepared tooth by filling it with an acrylic material that is then placed on the prepared tooth. This provides an acrylic temporary crown fitted to the prepared tooth that may be used while waiting for the fabrication of the final crown restoration.

It has been shown that the application of the hemostatic agent to the prepared tooth and tissue in the sulcular area prior to the application of impression with acrylic to the tooth, and a second application of hemostatic agent prior to placement of the temporary crown alleviates the need for the retraction step required in conventional procedures in the art. Accordingly, the temporary crown with its tissue contact and the hemostatic agent with its vasoconstricting effect allow for a clean and accurate impression without the need for retraction, which is normally a five-minute procedure.

When the permanent crown has been manufactured and shipped to the dentist, the patient returns to the office of the dentist for removal of the temporary crown and installation of the permanent crown at block 150 of FIG. 1. Removal of temporary crowns and adhesive and the trial placement of the permanent crown or veneer can cause the bleeding resulting in contamination of the tooth surface, particularly at the base of the tooth. Blood, saliva and adhesive remnants must be removed from the tooth surface prior to the fitting and installation of the permanent crown.

Optionally, a thorough coronal cleaning of the contoured teeth is performed to remove the salivary pellicle and any calculus or foreign matter that may be present at block 160 of FIG. 1. In one embodiment, the enamel surfaces are polished with an oil or residue free polish to facilitate penetration of the acid etchant into the enamel at block 170. A conventional bristle brush or rubber polishing cup may be used on tooth surfaces with polishing compound along with a pressurized water and air rinse to optimize the effect of the etchant on the surfaces to be bonded with a prosthetic. The optional preliminary cleaning can be particularly beneficial to facilitate adhesion of portions of the tooth surface that are not directly modified to receive the prosthesis. Occasionally, it will be necessary to remove some of the temporary adhesive with a dental burr prior to polishing the tooth surface.

Insufficient cleaning or removal of polishing compounds, burr and enamel debris can reduce the strength of the bond between the tooth surface and the dental or orthodontic restoration. In addition, the tissues that are adjacent to the tooth base are often inflamed due to the presence of the temporary crown or veneer and will bleed with minimal stimulation from rinsing with pressurized water and air. The proteinaceous surface coating of blood etc. will decompose over time if it is not removed and will permit microleakage to occur at the junction between the tooth and the adhesive allowing the seal to be broken and fluid to flow underneath the restoration. Such leakage may result in secondary cavities or a failure of the bond of the prosthesis to the tooth surface.

Once the tooth surface is cleaned and prepared after removal of the temporary, an etchant composition is applied at block 170 of FIG. 1. The strength of the bond between the contoured tooth and the crown or veneer can be substantially increased with the use of an etchant. The tooth that is to be etched is preferably isolated using lip or cheek retractors and cotton rolls as necessary to provide a dry tooth surface area to apply the etchant composition. Typically, the preparatory activities and the fitting of the prosthesis cause recurrent bleeding in the gingival spaces and on to the tooth surfaces. The tooth surfaces that are to be etched should be clean and dry before the application of the etchant composition at block 170.

The etchant composition according to the present invention generally comprises an etchant in combination with one or more hemostatic agents alone or in combination with an anti-fibrinolytic agent to control and eliminate the presence of blood during the preparation, etching and fitting activities. Elimination of the presence of blood will permit proper etching at block 170 and subsequent bonding with adhesive at block 180 of FIG. 1.

Etching of enamel surfaces with an etchant such as orthophosphoric acid has been used to increase bond strength between teeth and composite resin adhesives for many years. It has been shown that phosphoric acid concentrations of between 30-40% applied to tooth surfaces for 15 to 60 seconds preferentially removes prism core material in enamel as well as remove the smear layer produced during preparation of the tooth for the dental restoration. The application of concentrations of greater than approximately 50% phosphoric acid results in the appearance of monocalcium phosphate monohydrate, which is not easily removed from the tooth. Similarly, the application of an etchant to dentin surfaces for approximately 15 to 30 seconds can open dentin tubules and cause demineralization of the peritubular and intertubular dentin as well as the removal of the smear layer. The selective dissolution of prism cores or peripheries creates micropores and enamel rods in the surface of the enamel that resin can flow into and polymerized to form a mechanical bond with the enamel.

It will be seen that the amount of surface etching and demineralization of the dentin and enamel will depend on the type etchant that is used, the concentration of the etchant and the time of exposure to the etchant. While an etchant of approximately 30% to approximately 40% phosphoric acid is preferred, other etchants such as 10% maleic acid for 15 to 60 seconds may also be used.

The etchant is combined with at least one hemostatic agent. In one embodiment, a dual or triple chambered syringe with a mixing nozzle is used with the etchant stored separately from the hemostatic agents until use. In another embodiment, the etchant and hemostatic agent are pre-mixed and administered with a single chambered syringe. The etchant and hemostatic agent may be in gel or liquid form. The etchant and the hemostatic agents may also include a colorant that can act as an indicator of the presence of the composition on the teeth and assist the dental practitioner in providing complete coverage of the tooth. For example, the etchant may have a blue color and the hemostatic agents have a red color. The combination of the two colors produces a third color in this embodiment.

Hemostatic agents that may be used in combination with the etchant may be used alone or in combination with other agents including topical vasoconstrictors, coagulation promoters or astringents that will restrict or eliminate blood flow or cause contraction of gingival tissues.

In the preferred embodiment, the hemostatic agent comprises a topical solution of epinephrine. One embodiment provides a mixture with concentrations of approximately 0.5 mg to 1.0 mg epinephrine per 1.0 ml etchant solution. Another embodiment provides a preparation ranging between approximately 5 mgs and approximately 10 mgs of epinephrine per milliliter of topical solution. Yet another embodiment uses approximately 1.0 mg to approximately 10.0 mg of epinephrine per 1.0 ml etchant solution.

In use, a preparation of epinephrine solution with 10 mg/ml strength, for example, approximately 0.2 ml of the solution would be applied to the tissue around the effected tooth. With a 0.2 ml application, the capillaries responsible for bleeding would be able to absorb the entire 0.2 mg of epinephrine that is present. The preferred ratios of epinephrine to total solution range from 1:2000 to 1:10,000.

It will be seen that even total absorption of the administered dose of the preparation is at a very safe level. The hemostatic effect is to temporarily restrict or stop capillary flow, thereby disallowing any further absorption and bleeding keeping absorption to a minimum. Therefore, relatively strong doses can be applied to the teeth and gums and excessive absorption of the hemostatic agent epinephrine can be avoided.

In another embodiment, thrombin is provided as a haemostatic agent that can be used in combination with the etchant. Thrombin proteolytically cleaves fibrinogen into fibrin, which crosslinks in a wounded area to control bleeding. The preferred concentration of thrombin in the composition ranges from approximately 100 units to approximately 500 units of thrombin per milliliter of composition solution.

Another embodiment includes an antifibrinolytic agent alone or in combination with a hemostatic agent to control gingival bleeding by topical application. The preferred concentration of aminocaproic acid is approximately 125 mgs to approximately 250 mgs of aminocaproic acid per milliliter of composition.

In one embodiment, a paste is provided having approximately equal amounts of epinephrine and aminocaproic acid. For example, the dental practitioner can compose the paste by combining 0.5 g of conventional gel powder with 2 ml of glycerin. Added to the paste are 30 ml of epinephrine and 30 ml of aminocaproic acid and then mechanically mixed into the paste. Gelling agents may include Sodium Alginate or Tragacanth or other non-toxic gelling agent in this embodiment.

Other hemostatic agents such as naphazoline, tramazoline, phenylephrine, and cyclopentolate and other vasoconstrictors may also be used alone or in combination with the aforementioned hemostatic agents. The mechanism of blood flow reduction that is utilized by these agents may compliment the mechanisms of the other hemostatic agents that are used in the composition. It can be seen that hemostatic control can be efficiently achieved with a composition that influences multiple mechanisms and such control can be of sufficient duration that the installation procedure can take place without interference with gingival bleeding.

In another embodiment, the gel or liquid etchant composition includes a colorant to allow the user to identify the location and relative quantity of the etchant that is applied to a tooth. The use of a colorant assists in complete coverage and an even application of etchant to each of the treated teeth. A colorant in the etchant composition may also act as an indicator of whether the etchant composition has been completely removed after exposure.

In another embodiment, the hemostatic agent has one color and the etchant is given a second complimentary color so that when the etchant and hemostatic agent are mixed a third color is produced. The third color provides a visual indicator that the etchant and hemostatic agent are mixed as well as an indicator that the combined material is properly applied or removed in this embodiment. The color may also serve as a visual indicator of the relative quantity of material that has been applied on the surface of the substrate to assist in the even or complete distribution of material.

The colorant may also be useful with applications of hemostatic agents alone. For example, hemostatic agents may be applied to pulpal tissues to control bleeding. Removal of extensive caries or replacement of an existing filling can result in the encroachment into the pulpal space. The pulpal tissue, which is the nerve and blood supply for the tooth, often responds negatively to such encroachments resulting in symptoms that require root canal treatments. However, if the pulpal space exposure is small (less than approximately 2 mm), and bleeding can be controlled, a glass ionomer liner can be placed, and the symptoms will frequently disappear without the need for a root canal treatment. The hemostatic agent of the present invention works well in this control process, and allows the placement of a liner.

It has also been observed that the pH of the etchant composition may be raised by the dilution of phosphoric acid during the combination with some hemostatic agents and during use in the mouth of the patient. In one embodiment, appropriate buffers are optionally added to help maintain an optimal pH for the activity of the etchant. Acid-base buffers typically consist of a weak acid and its conjugate base.

At block 180 of FIG. 1, the etchant composition is preferably removed and the etched surface dried prior to the application of adhesive. The treated surface is preferably washed with water and the excess water removed with air. Wiping of the surface of the tooth should be avoided because wiping will fracture the enamel rods created during etching, and remove fibrils of collagen from the dentinal tubules that are exposed in the demineralization process and thereby weaken bond strength. Saliva should not be allowed to come in contact with the prepared enamel after the etchant application and the washing and drying of the tooth surfaces. Saliva contains proteins that will permit the enamel to remineralize during process of bonding and bacteria will coat the dentin/enamel surfaces introducing an unwanted layer at the adhesive/tooth surface. Accordingly, directing air from the top of the tooth toward the gingiva during drying may prevent subgingival saliva from contacting the enamel. Enamel contaminated with saliva may be re-etched for a period of time, preferably ten seconds or less, and then rinsed and dried a second time.

Care should also be taken not to leave the phosphoric acid etchant on the surface of the tooth for more than approximately 90 seconds because it will result in the formation of insoluble calcium phosphate crystals that cannot be removed by rinsing. Over etching will also reduce the bond strength of the adhesive.

At block 180, the adhesive may be applied to the prepared tooth and to the sized prosthesis. The prosthesis is then positioned and permanently installed.

Referring now to FIG. 2, one method 200 adapted for installation of a veneer is shown. Porcelain or other ceramic veneers are thin walled prosthetics that maintain a stable color and are very durable. Veneers may be appropriate for stained, broken or chipped teeth as well as for the elimination of gaps between teeth or for misaligned teeth. Similar to crown placement, the installation of a veneer includes the removal of a very thin layer of tooth structure from the outer surfaces of each tooth that is receiving a veneer at block 210 of FIG. 2. An impression of the prepared surfaces of each tooth is preferably taken at block 220 so that veneers with customized fitted surfaces may be manufactured at a dental laboratory at block 230 of FIG. 2.

In veneer installations the prosthesis is preferably placed on the prepared tooth before the application of adhesive or etchant to check the color match as well as the overall appearance. The materials used in restorative dental veneers and other treatments may be translucent, which helps give the treated teeth a lifelike look. Because the materials of the veneer are translucent, the color and substructure of the underlying natural tooth may influence the final shade of the restoration.

Various shades of cements may be used to achieve the subtle variations in color of the installed veneer to ensure an even and natural shade to the permanent veneer restoration. Shaded pastes that match the final cements may be applied to the restoration to eliminate the guesswork from the final outcome at block 240 of FIG. 2. The shaded “try-in” pastes allow the dental practitioner to accurately observe the final shade of the veneer before cementation, to assure the proper final permanent result. Several shades of pigmented cement may be necessary for any given procedure. For example, the placement of veneers on the front teeth of a patient from canine to canine may require three or more different shades of cements to achieve the right color shades for each tooth in the entire set.

It can be seen that even a small amount of blood on to the tooth surface where cement and bond resin is to be applied can result in discoloration as well as a weakened and porous bond when the surface is simply dried off with an air syringe prior to bonding. The iron in the heme group of hemoglobin has been shown to oxidize and “blacken” under the bonded cement surface and show through the restoration. Such discolorations may be in the form of spots or splotches or may subtly change the shade of the veneer restoration. Not only is the discoloration unsightly, blood residue may cause an interruption at the interface between the tooth and cement by the breakdown of an organic substrate and the seal of resin to tooth surface over time. This physical interruption at the interface can speed the failure of the restoration.

In one embodiment, a hemostatic agent is incorporated into the “try-in paste” used in the veneer or crown placement that helps streamline the cementation process. In this embodiment, hemostasis begins at the “try-in paste” stage at block 240 and then re-enforced at the etching stage at block 250 of FIG. 2, which is prior to the application of adhesive. This combined approach to hemostasis results in contamination free surfaces after etching as well as a superior bonding surface.

Preferably the provisional try-in pastes have colorants that are essentially identical to the shades of the adhesives. Once the veneer fit is adjusted and the shade approved, the veneer is removed and any excess paste removed from the surface of the tooth. The etchant composition with hemostatic agents is then applied to demineralize the tooth surface at block 250 of FIG. 2. Care should be taken not to allow blood, saliva or other contaminants to come in contact with the etched surfaces of the tooth. The adhesive resin that has been selected based on color is applied to the prepared surface of the tooth at block 260 of FIG. 2 and the veneer is placed and set into place at block 270.

Accordingly, it can be seen that a composition and method of use is provided that prepares and conditions a tooth surface for bonding with an adhesive while controlling gingival bleeding and contamination. A method and composition is also provided for controlling the presence of blood on tooth surfaces to be bonded with the use of a try-in paste with hemostatic agents.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A composition for preparing a tooth surface for adhesive bonding, comprising:
an etchant; and
a hemostatic agent.

2. A composition as recited in claim 1, wherein said hemostatic agent comprises epinephrine in the ratio ranging from approximately 1:2000 to approximately 1:10,000 of etchant to epinephrine.

3. A composition as recited in claim 1, wherein said hemostatic agent comprises thrombin with a concentration ranging from approximately 100 units to approximately 500 units per milliliter of composition.

4. A composition as recited in claim 1, wherein said hemostatic agent comprises aminocaproic acid with a concentration ranging from approximately 125 mg to approximately 250 mg per milliliter of composition.

5. A composition as recited in claim 1, wherein said etchant comprises approximately 30% to approximately 40% phosphoric acid by volume.

6. A composition as recited in claim 1, wherein said etchant comprises approximately 10% to approximately 15% maleic acid by volume.

7. A composition as recited in claim 1, wherein said etchant includes a colorant.

8. A composition as recited in claim 7, wherein said hemostatic agent includes a colorant.

9. A composition as recited in claim 1, further comprising an antifibrinolytic agent.

10. A composition as recited in claim 9, wherein said antifibrinolytic agent comprises aminocaproic acid.

11. A composition as recited in claim 10, wherein said antifibrinolytic agent comprises aminocaproic acid with a concentration ranging from approximately 125 mg to approximately 250 mg per milliliter of composition.

12. A composition as recited in claim 1, further comprising a buffer, wherein said buffer maintains the approximate pH of the composition.

13. A topical surface conditioner composition for improving adhesive bonding with a dental or orthodontic appliance, comprising:
an etchant;
a first hemostatic agent; and
at least one second hemostatic agent.

14. A composition as recited in claim 13, wherein said first hemostatic agent comprises epinephrine.

15. A composition as recited in claim 14, wherein said first hemostatic agent comprises epinephrine in a ratio ranging from approximately 1:2000 to approximately 1:10,000 of etchant to epinephrine.

16. A composition as recited in claim 13, wherein said second hemostatic agent comprises thrombin.

17. A composition as recited in claim 16, wherein said second hemostatic agent comprises thrombin with a concentration ranging from approximately 100 units to approximately 500 units per milliliter of composition.

18. A composition as recited in claim 13, wherein said second hemostatic agent comprises an antifibrinolytic agent.

19. A composition as recited in claim 18, wherein said antifibrinolytic agent comprises aminocaproic acid.

20. A composition as recited in claim 19, wherein said antifibrinolytic agent comprises aminocaproic acid with a concentration ranging from approximately 125 mg to approximately 250 mg per milliliter of composition.

21. A composition as recited in claim 13, wherein said etchant comprises approximately 30% to approximately 40% phosphoric acid by volume.

22. A composition as recited in claim 13, wherein said etchant comprises approximately 10% to approximately 15% maleic acid by volume.

23. A composition as recited in claim 13, further comprising an antifibrinolytic agent.

24. A composition as recited in claim 23, wherein said antifibrinolytic agent comprises aminocaproic acid.

25. A composition as recited in claim 23, wherein said antifibrinolytic agent comprises aminocaproic acid with a concentration ranging from approximately 125 mg to approximately 250 mg per milliliter of composition.

26. A composition as recited in claim 13, further comprising a colorant.

27. A composition as recited in claim 13, further comprising a buffer, wherein said buffer maintains the pH of the composition.

28. A topical surface conditioner composition for improving adhesive bonding with a dental or orthodontic appliance, comprising:
an etchant;
a hemostatic agent; and
an antifibrinolytic agent.

29. A composition as recited in claim 28, wherein said etchant comprises approximately 30% to approximately 40% phosphoric acid by volume.

30. A composition as recited in claim 28, wherein said etchant comprises approximately 10% to approximately 15% maleic acid by volume.

31. A composition as recited in claim 28, wherein said hemostatic agent comprises epinephrine.

32. A composition as recited in claim 31, wherein said hemostatic agent comprises epinephrine in a ratio ranging from approximately 1:2000 to approximately 1:10,000 of etchant to epinephrine.

33. A composition as recited in claim 28, wherein said hemostatic agent comprises thrombin.

34. A composition as recited in claim 34, wherein said hemostatic agent comprises thrombin with a concentration ranging from approximately 100 units to approximately 500 units per milliliter of composition.

35. A composition as recited in claim 28, wherein said antifibrinolytic agent comprises aminocaproic acid.

36. A composition as recited in claim 28, further comprising a colorant.

37. A method for preparing tooth surfaces for adhesion of dental or orthodontic prosthesis, comprising:
contouring a dental surface to receive a prosthesis; and
etching said contoured surface with an etchant, a hemostatic agent and an antifibrinolytic agent.

38. A method as recited in claim 37, wherein said hemostatic agent comprises epinephrine.

39. A method as recited in claim 37, wherein said hemostatic agent comprises epinephrine in a ratio ranging from approximately 1:2000 to approximately 1:10,000 of etchant to epinephrine

40. A method as recited in claim 37, wherein said hemostatic agent comprises thrombin.

41. A method as recited in claim 37, wherein said hemostatic agent comprises thrombin with a concentration ranging from approximately 100 units to approximately 500 units per milliliter of composition.

42. A method as recited in claim 37, wherein said antifibrinolytic agent comprises aminocaproic acid.

43. A method as recited in claim 37, wherein said antifibrinolytic agent comprises aminocaproic acid with a concentration ranging from approximately 125 mg to approximately 250 mg per milliliter of composition.

44. A method as recited in claim 37, wherein said hemostatic agent and said antifibrinolytic agent are provided in equal concentrations.

45. A method as recited in claim 37, further comprising:
applying an evaluation paste with a colorant to said etched surface;
comparing the color of a prosthesis and said evaluation paste with adjoining teeth; and
selecting a colored adhesive that is approximately the same shade as the evaluation paste.

46. A method as recited in claim 45, wherein said evaluation paste includes at least one hemostatic agent.

47. A method as recited in claim 46, wherein said hemostatic agent comprises epinephrine.

48. A method as recited in claim 46, wherein said hemostatic agent comprises thrombin.

49. A method as recited in claim 46, wherein said hemostatic agent comprises a combination of thrombin and epinephrine.

50. A method as recited in claim 46, wherein said hemostatic agent further comprises a combination of thrombin and epinephrine and an antifibrinolytic agent.

51. A method as recited in claim 50, wherein said antifibrinolytic agent comprises aminocaproic acid.

52. A method as recited in claim 37, further comprising:
polishing said contoured surface prior to etching.

53. A paste for the evaluation of the color and position of a dental prosthesis prior to bonding, comprising:
a paste; and
at least one hemostatic agent.

54. A composition as recited in claim 53, further comprising:
at least one colorant, wherein the color of an adhesive and prosthesis can be evaluated before permanent installation of the prosthesis.

55. A composition as recited in claim 53, wherein said hemostatic agent comprises a vasoconstrictor or an antifibrinolytic agent.

56. A composition as recited in claim 53, wherein said hemostatic agent comprises an agent selected from the group of agents comprising: epinephrine, thrombin and aminocaproic acid.
